(19) United States
(12) Patent Application Publication
(10) Pub. No.: US 2021/0292693 A1
(43) Pub. Date: Sep. 23, 2021

(54) BIOGAS PRODUCTION BY MEANS OF MULTI-STAGE FERMENTATION IN A MONO-TANK

(71) Applicant: Planungsbuero Rossow - Gesellschaft Fuer Emeuerbar e Energien MbH, Neubrandenburg (DE)

(72) Inventor: Norbert ROSSOW, Penzlin (DE)

(21) Appl. No.: 14/649,773

(22) PCT Filed: Dec. 9, 2013
(86) PCT No.: PCT/EP2013/075994

§ 371 Date: Jun. 4, 2015

(30) Foreign Application Priority Data
Dec. 7, 2012 (DE) 10 2012 222 590.0

(51) Int. Cl. C12M 1/107; C12M 1/00; C12M 1/34; C12M 1/36

(52) U.S. Cl. CPC C12M 21/04; C12M 23/34; C12M 29/00; C12M 41/12; C12M 41/48; C12M 23/36

(57) ABSTRACT

The invention relates to the production of biogas by means of multi-stage fermentation in a mono-tank. The device for biogas production by means of multi-stage fermentation in a single tank according to the invention is characterised in that the inside of the tank is provided with additional partitions, which are arranged in such a manner that the total volume of the tank is divided into at least two or more partial volumes, and partitioned tank sections are thus present, in which the various fermentation stages occur and which can be controlled according to the actual reaction progression. The device optionally has an ultrasonic module for treating a recirculated material from an advanced fermentation stage or from the device according to the invention.

### DESCRIPTION

The invention relates to the production of biogas by means of multi-stage fermentation in a mono-tank.

### STATE OF THE ART

The generation and utilization of biogas through anaerobic fermentation of the organic components of biomass and animal excrement as an alternative to the use of finite conventional energy sources is becoming increasingly important. However, its advantage can be counteracted by heightened and rising costs for the construction of biogas plants in light of higher raw material prices and in the context of regulatory requirements to be implemented with regard to plant safety and environmental compatibility.

In most cases put into practice, biogas plants consist of one or more insulated and heated fermenter tanks in which organic dry matter is converted by way of a wet fermentation process using microbacterial methanogens for the generation of biogas from suitable substrates.

This process may be preceded by devices for crushing the input materials and dosing units for mixing. More frequently, this mixture is subsequently pretreated in separate heated hydrolysis tanks over a certain period for the purpose of further fermentation and it is only thereupon added into the fermenters.

Depending on the situation, additional tanks or lagoons follow downstream from the fermenters for secondary fermenting or uptake of the digestate. The biogas generated does not always accumulate continuously and is not always passed on or processed further, which is why flexible gas tanks are provided. These can be separate gas tanks or be provided on one or more roofs of the aforementioned tanks. Otherwise, all tanks have gas-tight covers.

In order to ensure and optimize the fermentation processes, the individual tanks are equipped with agitators or other options for mixing. The addition or treatment, collection or discharge of fermentation material, gas, condensate, water and electricity requires various piping systems, pumps, fittings, etc., which, alone, make for the complexity characteristic of a functional biogas plant. Furthermore, measuring, testing, control and regulator units are indispensable for this purpose.

As part of the efforts to increase the effectiveness of biogas fermentation, it has been proposed to enlarge plant dimensions. Accordingly, document WO 2005/054423 proposed large-scale fermenters for the generation of biogas from biomass, and stacking renewable raw materials containing high concentrations of dry substance in one or more hall-like tanks, and continuously moistening them with percolate. Similarly, the process described in document DE 10 2007 029 700 comprises a multitude of fermenters of the garage type, however, likewise also not expressly for wet but rather for dry fermentation.

In documents DE 197 46 636, DE 10 2007 005 069 or DE 10 2009 059 262, solutions are suggested, for which one or more smaller tanks are respectively integrated into one or more larger tanks.

Patent specification US 005637219 A is also included in the state of the art. Said patent specification concerns a horizontally perfused and likewise horizontally reacting reactor system, which is not divided by partitions that are firmly connected to the exterior wall. It relates to a rotating system with integrated agitator elements, which are mounted fixed in the rotation body. A closed tank is concerned in the cited document, which is used for the process steps and the reaction fluid is combined in the complete tank, whereby this fluid must be regarded as a homogeneous fluid. The gas is discharged without previous collection of the gas in a flexible membrane tank. The fluid is discharged without being able to fill or empty the sections separately from each other.

Disclosures US 2003/00334300A1 and WO 2008/099227A1 concern closed mono-reaction chambers in which the reaction fluid, from the entry up to the exit point, originates from the same source and wherein a horizontal media flow must take place for the intended reaction. The sections of the tank are suitable to produce completely independent reactions without one section necessarily being reliant on the volume flow or the reaction in the other section chamber.

The cited documents also do not include any gas chambers directly connected to the tank wall, independent of the sections in the individual or collective gas tank, and these are not designed as gas membrane tanks therein.

The disclosed technical solutions describe tanks in which a fluid flows from one entry to one exit, whereas the sections are not independent from each other. In addition, an upstream and downstream flow is required in these tanks for the function, meanwhile, this flow does not take place in our own invention as described further below and is not required for the function.

### PROBLEM TO BE SOLVED BY THE INVENTION

The invention has the underlying purpose of providing a further simplification of the technical framework conditions for the fermentation processes and limiting the number of tanks to be used for the respective specific process states to a single mono-tank.

### SOLUTION TO THE PROBLEM

The problem has been solved according to the characteristics of the patent claims.

According to the invention, expenditure was reduced to the necessary minimum to make multi-stage operation of biogas plants more affordable and, additionally, to make continuous operation more effective in terms of business management. Different stages in fermentation are concentrated according to the invention and take place in one single tank. Process stages such as hydrolysis, fermentation to acetic acid, CO₂ and H₂, and fermentation to methane are separated in the process. The multi-stage layout of fermentation results in greater material utilization of the substances used. Fermentation conditions are optimized in spatially-separated tank segments. The dwell time in the respective reaction tank is controlled as dependent on the progress of the reaction.

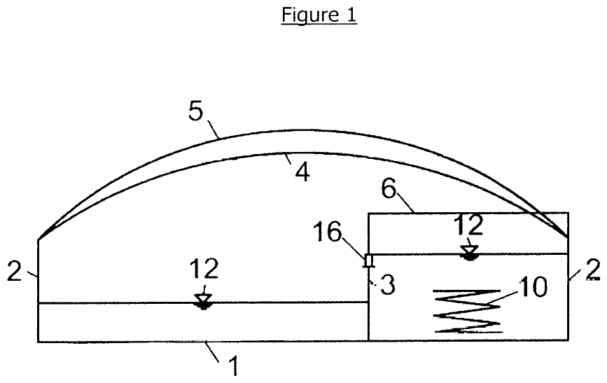

For this purpose, the tank body, as shown in FIG. 1, comprising the floor (1) the self-contained tank exterior wall (2) immediately adjacent to the floor or vertically mounted on it, which can be conceivably designed in a continuous cylindrical shape with a vertical cylinder axis, is provided with additional partitions (3) on the inside and, through the arrangement of which, the total volume of the tank body can be variably sectioned into at least two or more partial volumes. The spaces created by the additional partitions do not constitute independent tanks and are therefore also not produced from such.

In these separate spaces, specific environments are created in terms of feeding, heating, implementation and extraction, which correspond to various fermentation stages that are adjusted to each other or allow for such an adjustment. A reciprocal thermal effect is desired in individual cases.

The tank sectors generally possess an exterior wall area for one single tank that functions independently (mono-tank) and from this wall area, each individual reaction chamber can be fed, inspected, temperature-controlled—which is to mean heating or cooling—monitored and operated.

The height of the interior partitions is usually identical to that of the exterior wall, but it can also be lower or they can differ from each other. In the same way as the other parts of the mono-tank, they can be made of concrete, metal, stainless steel, plastic or compound materials, but they do not have to be identically composed to the other parts or be identical to each other in terms of their materials. The requirement is that the joints between the interior walls and the exterior wall are liquid-tight. The exterior wall is insulated against heat dissipation.

The tank body is variably provided across individual reaction chambers according to the state of the art, with a solid or membrane-like covering (4-6), which can be gas-tight or odor-inhibiting.

The general advantage derived from the invention is that the formerly multiple tanks of a biogas plant are substituted by one tank. Its dimensioning, which must necessarily be designed with greater height, is more than compensated for by the cost savings for the materials of the tanks that are no longer needed. Furthermore, economic benefits are generated from the outset by virtue of the pipe system that is far shorter and requiring fewer fittings, and lower pump capacities being required whereby smaller pumps are used and the assembly work and assembly time being reduced, etc. Compared to common biogas plants, the investment costs for construction are reduced by 30 to 40 percent. Due to a reduction of the floor sealing, the mono-tank, compared to a multi-tank biogas plant, is characterized by additional positive aspects in light of its better compatibility with land and nature, and a lower space requirement.

Additional advantages result in view of the biogas plant operation according to the specified purpose tor use.

Contrary to the state of the art described in documents US 005637219 A, US 2003/00334300A1 and WO 2008/099227A1, fill levels, reaction fluids, temperatures and volume flows that are completely independent from the neighbouring tank section area can be realized in the tank according to the invention.

According to the invention, the biogas plant can optionally be used for the fermentation of only one substrate (mono-fermentation) or one substrate mixture. If applicable, the substrate is fed into the mono-tank [prior to feeding into the mono-tank] by means of screw conveyor systems or other suitable materials handling equipment or, if applicable, it is homogenized by means of technical devices and, through the addition of fluid, it is conditioned so that can be conveyed by a pump and fed into the mono-tank in the further process.

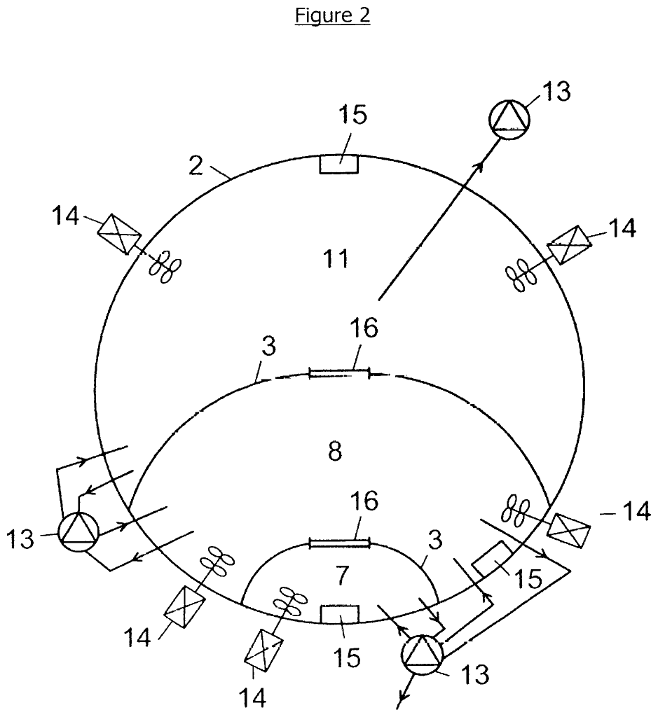

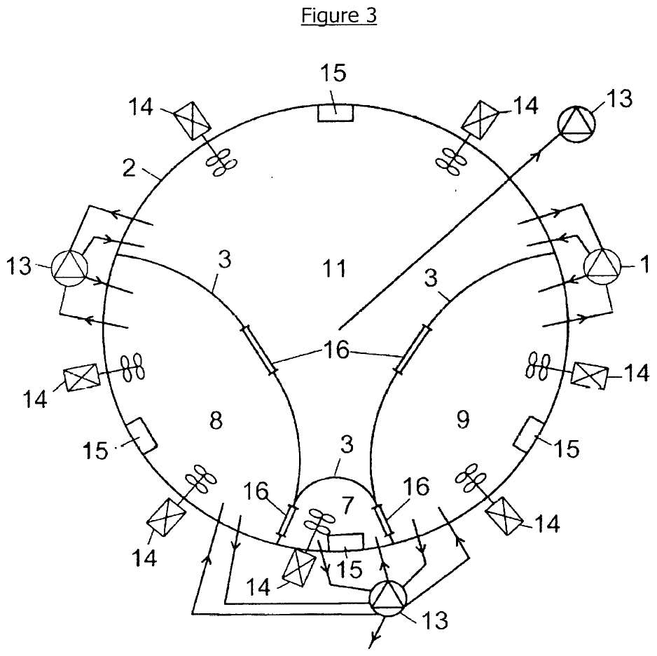

Here, this is initially a tank sector for hydrolysis (7), as shown in an example in FIG. 2 and FIG. 3, which can surely be designed in various forms in terms of its volume, but which is relatively small for technical reasons compared to the remaining functional tank sectors. In this tank sector, the fermentation material is mashed over a certain period, whereby it is “disintegrated” for the further fermentation process. Since the organisms effective in the fermentation process cannot directly absorb the macro-molecules of carbon hydrates, proteins and fats, e.g. poly-saccharides are hydrolyzed in the process from starch to oligosaccharides and monosaccharides, proteins to peptides and amino acids, fats to glycerin and fatty acids. As needed and beneficially, multiple hydrolysis chambers can also be embodied, which absorb different input at variable temperatures and dwell times.

After a sufficient period, the substrate provided with the products of hydrolysis reaches the next stage of fermentation, the actual fermentation, for which purpose a chamber (8) or multiple tank sectors (8, 9) can be structurally provided in the mono-tank with the same or different volumes. Throughout multiple phases and triggered, respectively, by the special strains of microorganisms, an acidification takes place through different bonds, at the end of which acetic acid predominates. It, in turn, is the basis for the metabolic processes of certain strains from the archaea group, in result of which methane is also formed, as well as other products, as a component of biogas, which is collected in the gas tank.

Both hydrolysis as well as fermentation can take place in different temperature ranges, whereas a mesophilic or thermophilic operation is preferable with regard to the biogas yield.

Therefore, the hydrolysis and fermentation chambers are optionally equipped with a shared or separate heater (10) on or in the floor or on or in the exterior walls.

The dwell time of the substrate in the fermenter and the related volumetric load fluctuates depending on the composition of the substrate. It is desirable that the greatest possible part of the organic dry substance available from the metabolic process is converted in the biogas formation process within the shortest possible time. This is not a linear process and the yield drops substantially near the end of the process.

The remaining digestate reaches one or multiple digestate sectors (11) of the mono-tank at the point in time that is suitable for this purpose. Because emptying depends on the season and is mostly done intermittently, the mono-tank will indicate different fill levels (12) in the same way as the other tank segments can easily have differing fill levels. In individual cases, the digestate tank can also be arranged externally or be supplemented by such.

The solution according to the invention as discussed here, presents significant advantages compared to traditional digestate tanks:

Based on complete insulation to the outside and elimination, in principle, of the transport path from the fermenters, there is a much lower temperature differential compared to the other functional chambers of the fermentation than is commonly the case. In consequence, the organic dry substance remaining in the digestate can also continue to be decomposed under relatively beneficial conditions. As the digestate stays in this tank for a relatively long period of time, secondary fermentation can be effected for substrate components, e.g. those containing cellulose, which otherwise largely escapes a metabolic process due to the short dwell time in the fermenter. The method according to the invention achieves a higher gas yield.

The required material transport or material exchange to, between and from the individual reaction chambers is realized by means of pump systems (13) via the shortest available passages and connections. Different fill levels in the individual tank sectors are reached, independent of the pump processes, through dam-like overflows (16) that are fixed or flexibly adjustable if tank sectors with identical gas pressure are provided. The development of optimal environments in the reaction chambers can be ensured by means of agitators (14) and the targeted triggering of mixing and flows. Multifunctional travelling shafts (15) permit access to the interior chambers of the fermenter during inspections for the purposes of repair or cleaning and, respectively, for adding or removing aggregates or devices.

The advantages that can be achieved in the ongoing operation in one mono-tank of multi-stage fermentation according to the invention—in addition to the cost savings already mentioned in the context of biogas plant construction—can be summarized as follows:

The complex insulation to the outside and greatest possible elimination of a thermal gradient between tank sectors in the interior saves a significant measure of thermal energy.

Short transport and pumping paths generate lower design requirements with respect to the required aggregates, decrease operating times, and minimize power consumption and the biogas plant's own electricity consumption.

Without greater additional expense, an effective secondary fermentation, continued fermentation of the digestate or the material to be discharged from the previously perfused tank sectors and/or fermentation stages is ensured under optimal process conditions.

Optionally, for the further optimization of material disintegration, an ultrasonic module is integrated into the preparation and hydrolysis system. This ultrasonic module was described in detail in the patent application submitted on the same date, under filing number DE 102013225322.2, and it is suitable for treating any fluids. In a special design variant, it can also be applied in combination with the mono-tank according to the invention. According to the invention, a multi-stage, self-regulating ultrasonic disintegration system is provided, which is not installed between or not externally in a separate tank, but which combines the required components and necessary elements in a compact design in one system for the direct attachment to, or installation in, the mono-tank, without requiring a separate building or container setup.

The ultrasonic module is comprised of the following elements:

system of pipes, which can also be square or rectangular in some areas,

pipe elements, pipe shutoff elements, measuring instruments,

test connections with equipment for testing, measuring and backwashing,

sonotrodes and integrated reflectors, fluid transporting units, and

backwashing units, fixtures and passage or connection equipment,

at least one reversible pump with rotation speed control.

The ultrasonic module transports the medium to be disintegrated from the mono-tank through pipe-like elements with an integrated transporting unit. It is mounted on or in the mono-tank. The fluid is transported on sonotrodes centrically integrated in the pipes via shutoff elements, pipe-work elements, volume flow-measuring devices and devices for mounting sensors and measuring elements, as well as the transporting unit, preferably in a vertical inflow.

The sonotrodes are coupled with matching reflectors, which are centrically arranged in the media flow at suitable spacing in parallel to the probe.

The system is designed so that the medium to be disintegrated is transported from the mono-tank to the disintegration probes.

The inflow takes place in a single or multi-stage process. In between the stages of disintegration, the effects from the individual disintegration nozzles can be assessed by means of integrated gauge connections and measuring elements. In addition, the viscosity and/or temperature, power consumption of the sonotrodes and the transporting unit can be measured. Depending on the measuring or analysis results, the system can activate further stages via the transporting unit (preferably a pump), whereby it is possible to increase the intensity (lower flow speed), reduce the intensity, or initiate backwashing.

The configuration in stages and the number of sonotrodes can be adjusted to the quantity and intensity of the disintegration. The integrated transporting units or devices can effect a counter-flow direction in order to, for example, perform backwashing. If necessary, the transporting unit can adjust the transporting unit capacity to needs/requirements (for example, rotation speed control).

The intake and flushing openings are secured against reciprocal effects through devices guiding the flow and, respectively, by the system's spatial arrangement.

The system is able to increase the effects and function by means of a system control unit, based on the communication between the setting and closing elements, the transporting unit, the measuring elements and the related analysis elements, the volume-measuring instrument, and the communication with any subordinate control or its own control.

It is even possible to install this system—with the exception of the transporting unit—within the fluid tank. All aforementioned components and required elements are combined in one system for direct attachment to, or installation in, the mono-tank. A separate building or container setup is not necessary.

By virtue of its design, this ultrasonic module is able to directly measure the effect from the sonication by means of the integrated control unit, as well as to modify and, if needed, adjust the intensity by means of the volume flow-control or the change of the flow direction (different passage of the fluid to the treated over a different number of sonotrodes).

Also, the self-cleaning function of the system, enabled through the reversal or change of the flow direction, as well as the possibility of increasing the volume flow and speed at the ratio 1:10, can be configured at regular intervals in the sonication system for prophylaxis.

The system can be equipped with common retail sonotrodes for in-pipe or on-pipe installation (thus, the sonotrodes are integrated both directly in the volume flow of the fluid to be treated as well as on the exterior wall of the pipe or in the exterior wall of the pipe).

Surprisingly, it became apparent that the wave-like shape of the piping of the ultrasonic system ensures, on the one hand, that the system is hydraulically optimized and, on the other hand, that a compact structural shape is achieved in observation of the spatial requirement for all components to be integrated. Through variation of the number of “waves,” the system can contain different numbers of sonotrodes or sonication areas, and it can thus be designed or built for differing sonication outputs.

The system can be installed on or in the mono-tank, as well as as a bypass system or inline system.

The advantages of combining the mono-tank with the ultrasonic module according to the invention are presented, for example, in that the investment costs for an ultrasonic module are lower by approx. 50% compared to present costs of about EURO 200 k. In addition, these systems also lower operating costs considerably, as the direct connection to the mono-tank reduces transport paths by multiples while they can furthermore be installed in a way that is beneficial for the flow and safe from clogging.

Furthermore, the ultrasonic module does not require any building-like enclosure; measures for insulation and protection against the weather, as for common piping installations, are sufficient.

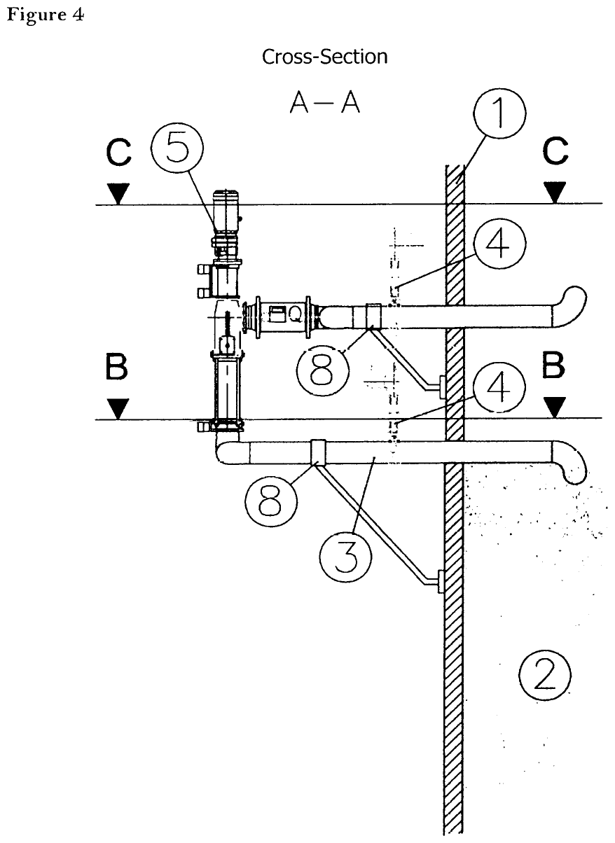

FIG. 4 shows a side view of the ultrasonic module. In this example, the ultrasonic module is mounted on the exterior side of the tank's inside wall. FIG. 2 shows a top view of the ultrasonic module. It can be seen that the ultrasonic treatment takes place directly in the substrate line—thus, no extra tank is necessary—and the sonotrodes are arranged either inside or outside the substrate lines. Likewise shown are the gauge connections or flushing nozzles and the sliders (pipe shutoff elements), as well as the pump (transporting unit).

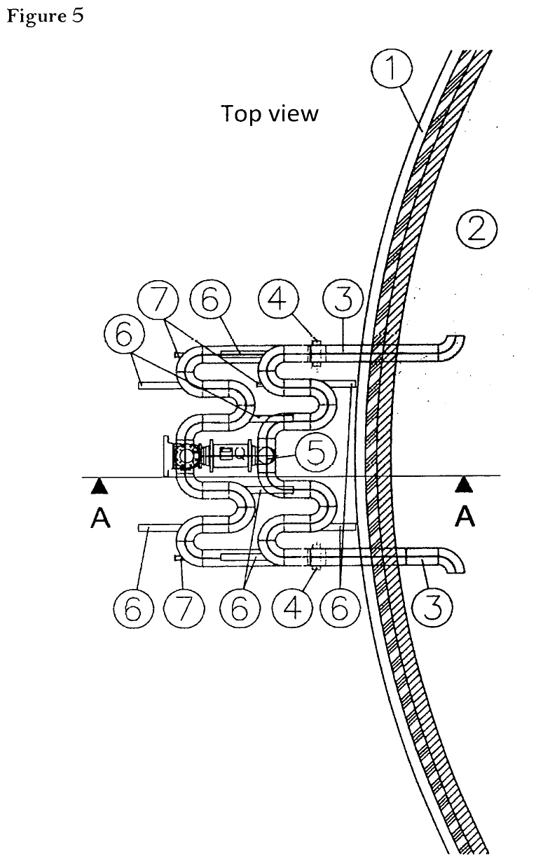

FIG. 5 also shows that a second stage can be connected to the ultrasonic treatment if necessary. The wave-like shape of the ultrasonic module, according to the invention, is shown particularly clearly in FIG. 2.

### REFERENCE LIST FOR THE MONO-TANK

| | | | |
|---|---|---|---|
| 1 | Tank floor | 2 | Exterior wall |
| 2 | Interior partition | 4 | Interior foil top |
| 5 | Exterior foil top | 6 | Fixed covering of chamber segment |
| 7 | Hydrolysis sector | 8/9 | Fermentation sector |
| 10 | Heater | 11 | Digestate sector |
| 12 | Fill level | 13 | Pump system |
| 14 | Agitator | 15 | Travelling shaft |
| 16 | Overflow | | |

### REFERENCE LIST FOR THE ULTRASONIC MODULE

1 Tank wall

2 Fermentation substrate

3 Substrate line

4 Slider

5 Reversible pump with rotation speed control

6 Sonotrude

7 Gauge connections and flushing nozzles

8 Support

1. Device for the generation of biogas through multi-stage fermentation in a single tank, characterized in that the tank is provided with additional partitions (3) on the inside, which, in turn, are arranged so that the total volume of the tank is sectioned into at least two or more partial volumes, whereby tank sectors are crated that neither are nor comprise an independent tank. In these tank sectors, the specific fermentation stages taking place can be controlled in terms of the reaction process and dwell time.

2. Device according to claim 1) characterized in that the single tank can have an exterior wall with variable height exterior wall areas.

3. Device according to claim 1) or 2) characterized in that the partial volumes of the tank can realize different fill levels.

4. Device according to claim 1) or 3) characterized in that differing temperatures can be realized in the partial volumes of the tank.

5. Device according to one of claims 1) to 4) characterized in that the joints between the interior partitions and the joints between these interior partitions and the exterior wall are liquid-tight.

6. Device according one of claim 1) or 5) characterized in that partial volumes can be covered differently with regard to the material, tightness and layer buildup, as well as the thermal insulation and be connected in the covering.

7. Device according one of claim 1) or 6) characterized in that the tank sectors contact the exterior wall from which each tank sector can be fed, inspected, temperature-controlled and individually operated.

8. Device according to one of claims 1) to 7) characterized in that it deliberately supports heat flow through the interior partitions in the interest of energy savings and that temperature differentials between neighbouring tank sectors are specifically utilized to support the processes of the specific fermentation stages.

9. Device according to one of claims 1) to 8) characterized in that either one or more hydrolysis chambers are integrated into the tank, which take up different input at variable temperatures, dwell times and volumetric loads.

10. Device according to one of claims 1) to 9) characterized in that either one or more fermentation chambers are integrated into the tank, which take up different input at variable temperatures, dwell times and volumetric loads.

11. Device according to one of claims 1) to 10) characterized in that it is additionally provided with a multi-stage, self-regulating ultrasonic module for the disintegration of re-circulating substrate fluid.

12. Device with ultrasonic module according to claim 11), whereas the ultrasonic module comprises the following elements:
a) at least one substrate line,
b) at least one transporting unit,
c) one or more sonotrodes,
d) device for the testing or measuring of fluid parameters,
e) device for connection to a tank,
characterized in that the sonotrodes are arranged inside of the substrate line or on the exterior side of the substrate line.

13. Device with ultrasonic module according to claim 11) or 12) characterized in that the substrate lines are arranged in a wave-like shape on the ultrasonic module and that a reversible pump with rotation speed control serves as the transporting unit.

14. Device with ultrasonic module according to one of claims 11) to 13) characterized in that the ultrasonic module is additionally provided with reflectors beside the sonotrodes, and comprising a control unit, which controls the pump by means of gauged parameters.
